(19) United States
(12) Patent Application Publication
(10) Pub. No.: US 2006/0025780 A1
(43) Pub. Date: Feb. 2, 2006

(54) Dual endoscopic polypectomy instrument arrangement

(76) Inventor: Oliver C. James, Shelbyville, KY (US)

Correspondence Address: DONALD N. HALGREN, 35 Central Street, Manchester, MA, 01944, US

(21) Appl. No.: 10/900,841

(22) Filed: Jul. 28, 2004

(51) Int. Cl. A61B 17/26

(52) U.S. Cl. 606/110

(57) ABSTRACT

A polypectomy instrument for the resection and withdrawal of a polyp tissue from a body lumen. The instrument comprises a dual handle arrangement on a first proximal end of the polypectomy instrument and a plurality of working channels on a distal end of the dual handle arrangement. Each of the working channels are arranged to carry a polyp engaging device. The dual handle arrangement is arranged to permit the easy manipulation and distally advancement and proximal retraction of the polyp engaging devices simultaneously.

### BACKGROUND OF THE INVENTION

1. Field of the Invention

This invention relates to medical devices and more particularly to a polyp resecting arrangement for the grasping and resecting of a polyp within the colon of a body.

2. Prior Art

A growth of a tissue mass protruding from a mucous membrane is called a polyp. Such mucus membrane may be found in the nose, the ears, the mouth, the lung, heart, stomach, intestines and the uterus and cervix. Typically, polyps are benign and eventually cease any growth. However, some polyps do keep growing, and may mutate to form a cancerous tumor. As such cancerous tumors grow, they burrow deeper into the tissue supporting that polyp. The cancer, in its final stages, invades the blood and lymph systems wherein any malignant cells of the tumor spread to other organs. Such a malignancy is severe in the colon. Such colon tumors however, may be readily treated.

Polyps grow from the mucus membrane with either a thin stalk or a broad base. A thin stalk holding a polyp is called a pedunculated polyp. A broad base which secures a polyp to the membrane, is called a sessile polyp.

U.S. Pat. No. 6,015,415 to Avellanet shows a polypectomy snare instrument which used as a snare loop connected to a power source to supply cautery current. The snare loop captures the polyp. A current is then supplied to the snare loop to cauterize the polyp as the snare is closed tighter around that polyp stalk. A pair of graspers would then be introduced through the sheath after the snare has been removed therefrom. The graspers would seize upon the polyp and remove it from the colon by withdrawal into the distal end of the tubular sheath carrying their instrument. Such instrument would then be removed. Such a procedure unfortunately requires the insertion and removal of multiple instruments through multiple sheaths. Each one consuming time and raising the problems of injury to the patient.

U.S. Pat. No. 5,746,747 to McKeating shows a polypectomy instrument with a pair of parallel sheaths slidable adjacent one another to reach the site of a polyp. The snare rides down the outer circumference of the sheath of its adjacent grasper. Such an arrangement is very complicated and may likely become entangled with one another at their distalmost ends during the grasping and snaring process. U.S. Pat. No. 6,616,659 to de la Torre et al. shows a polypectomy device having a pair of reciprocably advanceable jaws. The jaws have on its distalmost end, a wire snare extending thereacross. Such a device permits the snaring and grasping of a polyp but minimizes the manipulability of the distalmost end of the instrument when both the snare and the jaws are fixed together.

It is an object of the present invention to overcome the disadvantages of the prior art.

It is a further object of the present invention to provide a polypectomy device which permits multiple arrangements to be utilized either sequentially or simultaneously with a common handle, providing minimum intrusion within the patient.

It is still yet a further object of the present invention to provide a polypectomy device which minimizes the time and maximizes the device manipulability and the safety for the patient being operated upon.

### BRIEF SUMMARY OF THE INVENTION

The present invention comprises an endoscopic polypectomy instrument having a first or proximal end comprising a dual handle arrangement. The polypectomy instrument, in one preferred embodiment, has a second or distal end through which a biopsy forcep and a snare wire both simultaneously extend.

The dual handle portion of the instrument comprises an elongated generally cylindrical housing having an open ring at its proximalmost end as a thumb opening. The dual handle has an intermediately disposed frame portion with a distal end having a shaped distal frame grip thereon. The frame portion of the dual handle has a first slide member thereon having a pair of spaced apart flanges on either side of the handle with openings therethrough for insertion of the physician's fingers. The slide member is arranged to slide in an elongated track arranged in the intermediate portion of the dual handle instrument. The slide member is attached to the proximal end of a pair of push/pull control wires which extend through a first sheath. A pair of openable and closable jaws are arranged at the distal end of the first sheath. Distal movement of the slide member distally moves the control wires so as to spread apart the gripping jaws at the distal end of the first sheath. Proximal motion of the slide member with respect to the dual handle instrument pulls upon the control wires to close the jaws pivotably disposed on the distal end of the first sheath.

A snare wire slide control member is also arranged on the dual handle distal of the slide member at the distal end of the dual handle instrument. The snare wire slide control member is also movable both distally and proximally on the distal end of the dual handle instrument and between the slide member and the distal frame grip member arranged thereat. The snare wire control member is connected to a push/pull control wire having a distal end with a pre-shaped openable snare wire loop arranged thereat. The snare wire loop extends at the outer end of the second sheath. The snare wire is movable proximally so as to pull the snare wire loop tighter around a polyp if such a polyp has been located within a patient's colon.

The first sheath and a second sheath supporting the jaws and the snare loop respectively, are each individually and collectively movable reciprocably, in an outer conduit which extends through a colonoscope. The conduit is preferably attached to the distalmost end of the dual handle instrument distal of the frame grip member.

In one preferred embodiment of the present invention, the wire snare is electrified so that when it extends out of the distalmost end of the second sheath within the distal end of the conduit, the self-enlarging snare loop opens up and is positionable around a visualized colonic polyp, which then may be readily resected by closure of the snare wire loop about that polyp. Once the snare wire loop has resected the polyp, the jaws of the polypectomy instrument may be easily opened by distal movement of the slide member with respect to the dual handle instrument, so as to open the jaws and permit them to close and sieze upon the now resected polyp. Those closed jaws with the polyp tissue, may be withdrawn from colon with the colonoscope for subsequent pathological examination. The forceps jaws may be returned through the colonoscope, so as to retrieve any further polyp successively resected by the snare wire, if necessary.

The slide member may preferably have a circuit component therewith which is in electrical communication with an actuatable power source. The snare wire and the forceps may be both electrified to provide for cautery and bleeding control of any colon tissue. The actuable power source may be regulatable so as to permit either the forceps and/or the end or the snare wire loop to be electrified for such cautery and for bleeding control.

In yet a further preferred embodiment of the present invention, a retriever wire may be utilized with or instead of the biopsy forceps jaws so as to permit retracting a polyp with a three or four pronged finger arrangement.

The invention thus comprises a polypectomy instrument for the resection and withdrawal of a polyp tissue from a body lumen, comprising: a dual handle arrangement on a first proximal end of the polypectomy instrument; a plurality of working channels on a distal end of the dual handle, each of the working channels arranged to carry a polyp engaging device; and wherein the dual handle is arranged to distally advance, manipulate and proximally retract the polyp engaging devices simultaneously. At least one of the polyp engaging devices may have an electrical circuit connected thereto, for resecting and cauterizing a polyp thereby.

The dual handle arrangement preferably has a slide arranged therealong for permitting controlled motion of a forceps jaw arrangement therefrom. The dual handle also has a slide arranged therealong for permitting controlled motion of a wire snare loop arrangement therefrom. The dual handle may also have a slide arranged therealong for permitting controlled motion of a multi-pronged retriever wire arrangement therefrom.

The invention may also comprise a method of resecting and retrieving a polyp tissue from the body lumen of a patient being examined, comprising one or more of the following steps of: inserting a colonoscope into said body lumen of the patient; introducing a multi-working-channel polypectomy instrument through a conduit and into the colonoscope to permit internal visualization of the patient's colon, the polypectomy instrument having a dual handle to permit co-manipulating at least two instruments simultaneously therethrough; placing a wire snare loop around a located polyp on the lumen of the patient; resecting the polyp; grasping the resected polyp by a forcep arrangement manipulably disposed adjacent the wire snare loop in the polypectomy instrument; withdrawing the resected polyp with the colonoscope by withdrawing the forceps arrangement with the colonoscope. The resecting and grasping of the polyp preferably occurs simultaneously. The forceps may alternatively comprise a multi-pronged retriever wire device.

### BRIEF DESCRIPTION OF THE DRAWINGS

The objects and advantages of the present invention will become more apparent when viewed in conjunction with the following drawings, in which:

FIG. 1 is a side elevational view of an endoscopic polypectomy instrument constructed according to the principles of the present invention;

FIG. 2 is a view taken along the lines of A-A of FIG. 1.

FIG. 3 is a view taken along the lines of B-B of FIG. 1:

FIG. 4 is a view of the distal end of the instrument which view is taken along the lines C-C of FIG. 1;

FIG. 5 is a perspective view of the distal end of the polypectomy instrument showing both jaws and the wire loop snare extending therefrom;

FIG. 6 is a side elevational view of the first sheath of the polypectomy instrument showing the jaws in a closed orientation.

FIG. 7 is a view similar to FIG. 6 showing the jaws in an open configuration and,

FIG. 8 is a side elevational view of the polypectomy instrument showing an arrangement of grasper wires arranged adjacent to a snare in a sheathed conduit.

### DETAILED DESCRIPTION OF THE INVENTION

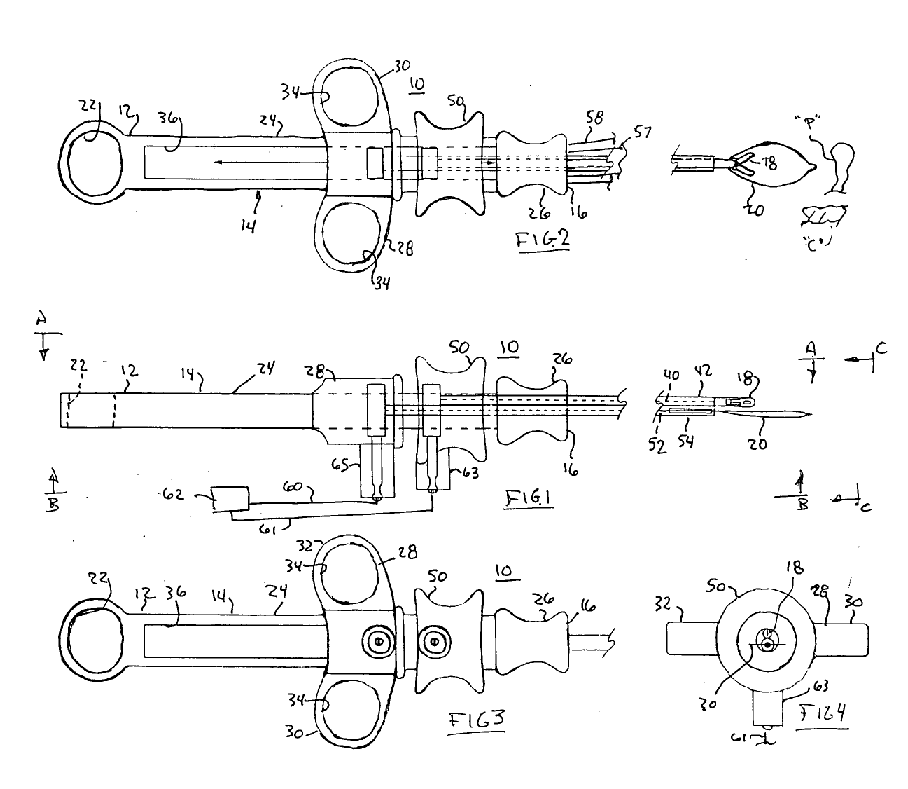

Referring now to the drawings in detail, and particularly to FIG. 1, there is shown the present invention which comprises an endoscopic polypectomy instrument 10 having a first or proximal end 12 comprising a dual handle arrangement 14. The polypectomy instrument 10, in a first preferred embodiment, has a second or distal end 16 through which a biopsy forcep 18 and a snare wire loop 20 extend.

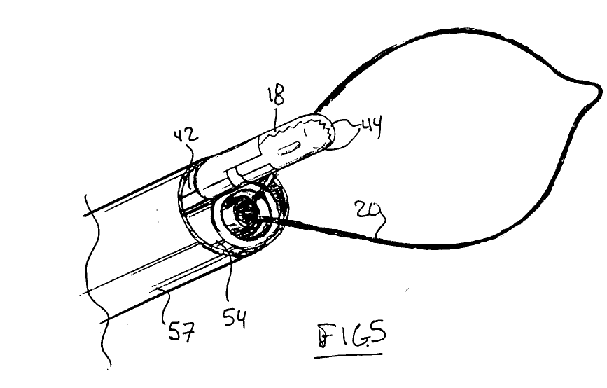

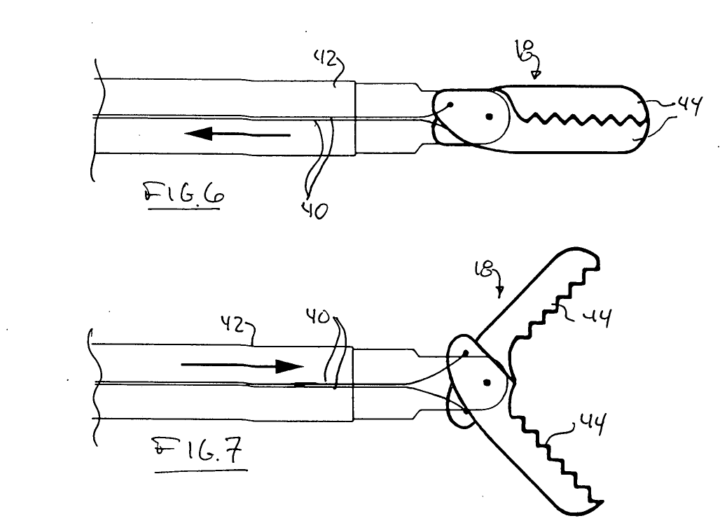

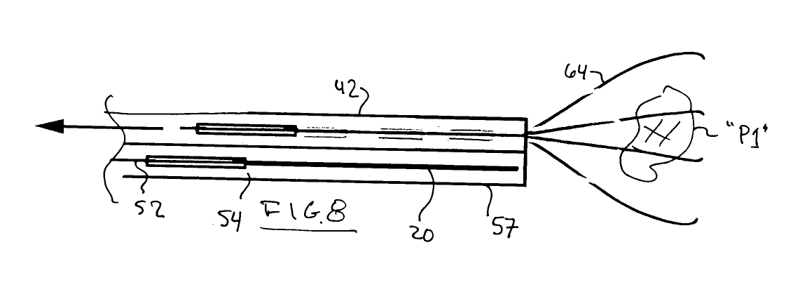

The dual handle portion 14 of the instrument 10 comprises an elongated generally cylindrical housing having an open ring 22 at its proximalmost end 12 as a thumb opening. The dual handle 14 has an intermediately disposed frame portion 24 with a distal end having a shaped distal frame grip 26 rigidly arranged thereon. The frame portion 24 of the dual handle 14 has a first slide member 28 thereon, as best show in FIGS. 2 and 3, having a pair of spaced apart flanges 30 and 32 on either side of the handle 14 with openings 34 therethrough for insertion of the physicians fingers. The slide member 28 is arranged to slide in an elongated track 36 arranged in the intermediate portion 24 of the dual handle instrument 10. The first slide member 28 is attached to the proximal end of a pair of push/pull control wires 40 which extend through a first sheath 42, as may be seen in FIGS. 5, 6 and 7. A pair of openable and closable jaws 44 are arranged at the distal end of the first sheath 42. Distal movement of the slide member 28 distally moves the control wires 40 so as to spread apart the gripping jaws 44 at the distal end of the first sheath 42. Proximal motion of the slide member 28 with respect to the dual handle instrument 14 pulls upon the control wires 40 to close the jaws 44 pivotably disposed on the distal end of the first sheath 42, as represented in FIG. 6.

A snare wire slide control member 50 is arranged distal of the slide member adjacent the distal end 16 of the dual handle instrument 14, as may be seen in FIGS. 1, 2 and 3. The snare wire slide control member 50 is also movable both distally and proximally on the distal end 16 of the dual handle instrument 14 and between the slide member 28 and the distal frame grip member 26 arranged thereat either simultaneously with or previous to the movement of the first slide 28 as commonly arranged in the dual handle 14 arrangement. The snare wire control member 50 is connected to a push/pull control wire 52, as represented in FIG. 1, having a distal end with its pre-shaped openable snare wire loop 20 arranged thereat. The snare wire loop 20 extends at the outer end of a second sheath 56. The snare control wire 52 is movable proximally so as to pull the snare wire loop 20 tighter around a polyp “P” if such a polyp has been located within a patient's colon “C”.

The first sheath 42 and the second sheath 54 supporting the jaws 18 and the snare loop 20 respectively, are each individually and collectively movable reciprocably, in an outer conduit 57 arranged within a colonoscope 58, having optic visualization means therein, not shown for clarity, which colonoscope 58 is positioned within the colon “C” of the patient being examined, as represented in FIG. 2. The conduit 57 and colonoscope 58 are preferably arranged to abuttingly engage the distalmost end of the dual handle instrument 10 distal of the frame grip member 26, as represented in FIG. 2.

In one preferred embodiment of the present invention, the wire snare loop 20 is electrified via a proper controlled electrical circuit 61 in hub 63 so that when it extends out of the distalmost end of the second sheath 54, which is electrically insulated within the distal end of the conduit 57, the snare loop 20 opens up and is positionable around a visualized colonic polyp “P”, which then may be readily resected by electrified or non-electrified closure of the snare wire loop 20 about that polyp “P”. Once the snare wire loop 20 has resected the polyp, the jaws 44 of the polypectomy instrument 10 may be opened by distal movement of the slide member 28 with respect to the dual handle instrument 10, so as to open the jaws 44 and permit them to close and seize upon the now resected polyp “P”, or the jaws 44 may have already been opened and grasped the polyp “P” prior to and in anticipation of its resection. Those closed jaws 44 and polyp tissue “P” may be pulled out with the colonoscope 58 for pathological examination of the polyp “P”. The forceps jaws 44 may be returned distally through the conduit 57, so as to retrieve a further any further polyp successively resected by the snare wire, if necessary.

The snare wire slide member 50 may also preferably have its circuit component 61 therewith which is in electrical communication with an actuatable power source 62, as represented in FIG. 1. The forceps 18 may alternatively both be electrified by a proper circuit 60, through hub 65 to therefore provide for simultaneous and/or sequential double cautery and bleeding control of any colon tissue through the readily manipulable common dual handle arrangement 14. The actuable power source 62 may be regulatable by a foot control member, not shown for clarity, by the attending physician, so as to permit either the forceps 18 and/or the end of the snare wire loop 20 to both be electrified for such cautery and bleeding control.

In yet a further preferred embodiment of the present invention, a multi-pronged retriever wire 64 may be utilized, as represented in FIG. 8, instead of the biopsy forceps jaws so as to permit retracting a larger resected polyp “PI” with such a hooked distal-ended three or four pronged retriever wire 64 arrangement instead of jaws to permit more appropriate manipulation of the retriever 64. Alternatively, a forcep arrangement and a wire retriever may be utilized side by side for any particularly problematical retrieval of a polyp.

I claim:

1. A polypectomy instrument for the resection and withdrawal of a polyp tissue from a body lumen, comprising:
a dual handle arranged on a first proximal end of said polypectomy instrument;
a plurality of working channels on a distal end of said dual handle, each of said working channels arranged to carry a polyp engaging device; and
said dual handle arranged to distally advance and proximally retract said polyp engaging devices simultaneously.

2. The polypectomy instrument as recited in claim 1, wherein at least one of said polyp engaging devices has an electrical circuit connected thereto, for resecting and cauterizing a polyp thereby.

3. The polypectomy instrument as recited in claim 1, wherein said dual handle has a slide arranged therealong for permitting controlled motion of a forceps jaw arrangement therefrom.

4. The polypectomy instrument as recited in claim 1, wherein said dual handle has a slide arranged therealong for permitting controlled motion of a wire snare loop arrangement therefrom.

5. The polypectomy instrument as recited in claim 1, wherein said dual handle has a slide arranged therealong for permitting controlled motion of a multi-pronged retriever wire arrangement therefrom.

6. A method of resecting and retrieving a polyp tissue from the body lumen of a patient being examined, comprising the steps of:
inserting a colonoscope into said body lumen of the patient to permit visualization of the colonic lumen;
introducing a multi-working-channel polypectomy instrument through said colonoscope to permit resection of a polyp found the patient;
placing a wire snare loop around a located polyp in the lumen of the patient;
resecting said polyp with said snare loop;
grasping said resected polyp by a forcep arrangement manipulably disposed adjacent to said wire snare loop;
withdrawing said resected polyp with said colonoscope by withdrawing said forcep arrangement with said colonoscope.

7. The method as recited in claim 6, including;
electrifying said snare loop as said resecting of said polyp occurs.

8. The method as recited in claim 6, including:
arranging a control member of said snare loop and a control member of said forcep arrangement on a dual handle for ease of manipulation.

9. The method as recited in claim 6, including:
electrifying said snare loop and said forcep arrangement simultaneously.

10. The method as recited in claim 6, wherein said resecting and said grasping of a polyp occurs simultaneously by manipulation of a pair of slide members on said dual handle arrangement.

11. The method as recited in claim 6, wherein said forceps comprises a multi-pronged retriever wire device.
